(19) United States
(12) Patent Application Publication
(10) Pub. No.: US 2016/0008533 A1
(43) Pub. Date: Jan. 14, 2016

(54) Feminine Hygiene System

(71) Applicant: Brenda Muhammad, Hopkinsville, KY (US)

(72) Inventor: Brenda Muhammad, Hopkinsville, KY (US)

(21) Appl. No.: 14/795,981

(22) Filed: Jul. 10, 2015

Related U.S. Application Data
(60) Provisional application No. 62/022,803, filed on Jul. 10, 2014.

(51) Int. Cl. A61M 3/02; A61M 39/22

(52) U.S. Cl. CPC A61M 3/0233; A61M 39/22; A61M 2210/14; A61M 2209/06

(57) ABSTRACT

A feminine hygiene system is provided. The feminine hygiene system is configured to allow a user to discretely transport a feminine hygiene product. The system includes a housing that resembles the shape of a purse forming an interior volume. The interior volume is configured to support a reservoir therein configured to support a volume of cleansing fluid therein. The housing includes an outlet valve that is in fluid communication with the reservoir, wherein a hose having an attachment end and a dispersing end is configured to removably attach to the outlet valve to dispense the cleansing fluid. The housing further includes a flap configured to provide access to the interior volume and a handle configured to allow a user to discretely carry the feminine hygiene system.

### CROSS REFERENCE TO RELATED APPLICATION

This application claims the benefit of U.S. Provisional Application No. 62/022,803 filed on Jul. 10, 2014. The above identified patent application is herein incorporated by reference in its entirety to provide continuity of disclosure.

### FIELD OF THE INVENTION

The present invention relates to feminine hygiene products. Specifically, the present invention provides a feminine hygiene system that allows a user to discretely transport feminine hygiene products by providing a reservoir within a housing configured to support a cleansing fluid therein, wherein the cleansing fluid can be dispensed via a hose attachment removably connected to an outlet valve of the housing.

### BACKGROUND OF THE INVENTION

Many women value their personal privacy and prefer not to advertise to others that they are carrying or using feminine hygiene products or feminine care products. For some women, openly carrying and using these products can lead to anxiety and trauma. Consumers often complain about the lack of discretion involved with carrying these feminine hygiene products to the restroom, or locating these items in a purse or carrying bag.

Particularly, conventional wrapping and packaging of these feminine hygiene products exacerbate the problem as these wrappings and packaging are well-known. Often, the problem is further compounded because the wrappings and packaging include vibrant colors and many distinguishing features that make the hygiene products easily recognizable. Furthermore, locating these products in a carrying bag or purse often can be distressing and difficult to disguise from those within the general vicinity. Therefore, there exists a need to provide a user with discrete means to disguise the carrying of these feminine hygiene products to avoid embarrassment and humiliation.

Devices have been disclosed in the prior art that relate to douche apparatuses having containment means. These include devices that have been patented and published in patent application publications. Some of these devices provide a vaginal hygiene system having a purse-like flexible case and a discharge tube, which is adapted to function as a vaginal douche. Other devices include a douche package that comprises a waterproof rubber bag, douche tips, and waterproof tubing. These devices, however, fail to provide a housing that is composed of waterproof material and an interior volume having a reservoir composed of plastic material.

The present invention provides a feminine hygiene product having a housing substantially shaped like a purse forming an interior volume. The interior volume is configured to support a reservoir therein, wherein the reservoir is configured to support a volume cleansing fluid therein. The reservoir includes an inlet valve configured to allow a user to fill and refill the reservoir with cleansing fluid. A lower end of the housing includes an outlet valve that is in fluid communication with the reservoir. A hose having an attachment end and a dispersing end is configured to removably attach to the outlet valve to disperse the cleansing fluid from the reservoir. An upper end of the housing includes a flap that is configured to enclose the interior volume and a handle. In this way, a user can discretely transport the feminine hygiene system to avoid embarrassment and humiliation.

It is therefore submitted that the present invention is substantially divergent in design elements from the prior art, and consequently it is clear that there is a need in the art for an improvement to feminine hygiene systems. In this regard, the instant invention substantially fulfills these needs.

### SUMMARY OF THE INVENTION

In view of the foregoing disadvantages inherent in the known types of feminine hygiene systems now present in the prior art, the present invention provides a feminine hygiene system wherein the same can be utilized for allowing a user to discretely transport feminine hygiene products, specifically a douching system.

It is therefore an object of the invention to provide a new and improved feminine hygiene system that has all of the advantages of the prior art and none of the disadvantages.

Another object of the present invention is to provide a new and improved feminine hygiene system comprising a housing having an interior volume configured to resemble a purse.

Yet another object of the present invention is to provide a new and improved feminine hygiene system, wherein said interior volume is configured to support a reservoir therein, wherein the reservoir is configured to support a volume of a cleansing fluid therein.

Still yet another object of the present invention is to provide a new and improved feminine hygiene system, wherein said reservoir includes an inlet valve configured to fill and refill the reservoir with cleansing fluid.

Yet a further object of the present invention is to provide a new and improved feminine hygiene system, wherein said housing includes an outlet valve configured to be in fluid communication with the reservoir.

Another object of the present invention is to provide a new and improved feminine hygiene system, wherein a hose having an attachment end and a dispersing end is configured to removably attach to said outlet valve to disperse said cleansing fluid within said reservoir.

Still yet another object of the present invention is to provide a new and improved feminine hygiene system wherein the device may be readily fabricated from materials that permit relative economy and are commensurate with durability.

Other objects, features, and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a front perspective view of the present invention.

FIG. 2 shows an overhead perspective view of the present invention.

FIG. 3 shows a front perspective view with a hose attachment of the present invention.

### DETAILED DESCRIPTION OF THE INVENTION

Reference is made herein to the attached drawings. Like reference numerals are used throughout the drawings to depict like or similar elements of the feminine hygiene system. For the purposes of presenting a brief and clear description of the present invention, the preferred embodiment will be discussed as used for discretely transporting feminine hygiene products, specifically a douching system. The figures are intended for representative purposes only and should not be considered to be limiting in any respect.

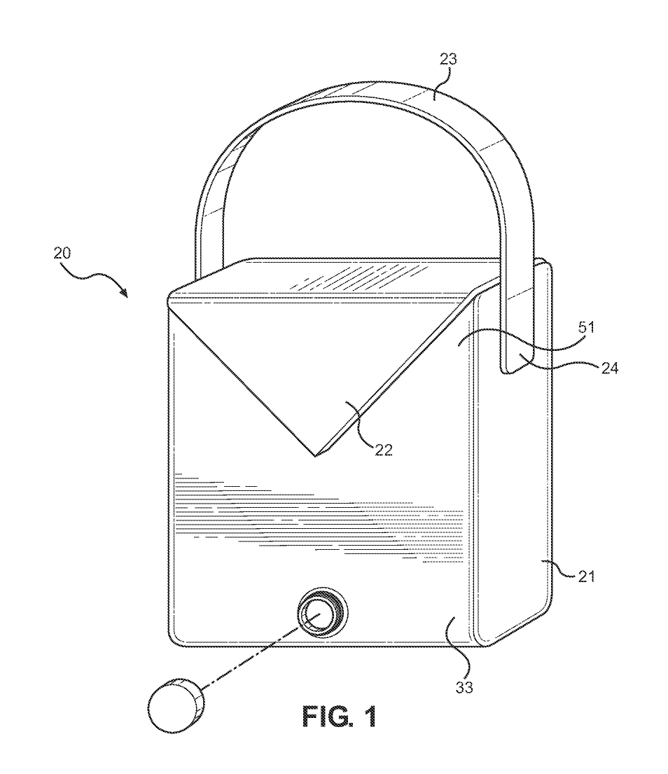

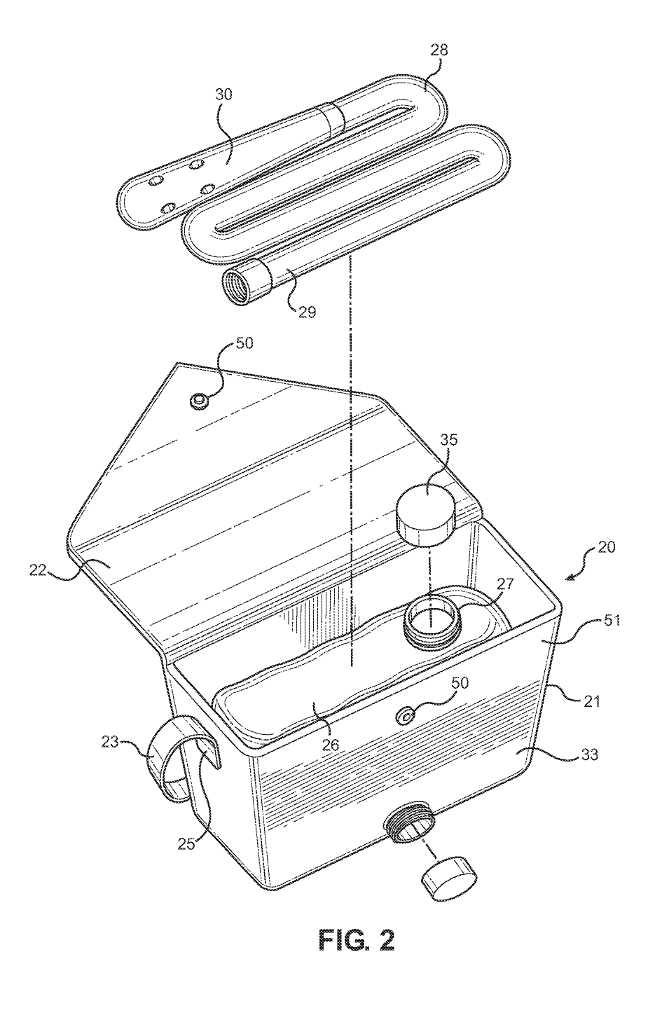

Referring now to FIGS. 1 and 2, there are shown a front perspective and an overhead perspective view of the feminine hygiene system 20, respectively. The feminine hygiene system 20 allows a user to discretely transport feminine hygiene products, specifically a douching system, freely without embarrassment. The feminine hygiene system 20 includes a housing 21 that forms an interior volume. The housing 21 is preferably composed from a waterproof material, such as nylon, however, other suitable waterproof material are also contemplated. The housing 21 is preferably rectangular in shape such that the housing 21 substantially resembles a purse. In other embodiments, the housing 21 is substantially shaped like a pocketbook or other accessories having an interior volume. These alterations and modifications to the housing 21 are deemed within the spirit and scope of the present invention and likewise contemplated.

The housing 21 includes a flap 22 on an upper end 51 that provides access to the interior volume. As illustrated, the flap 22 is substantially V-shaped, however, other shapes and sizes to the flap 22 are suitable and likewise contemplated. In some embodiments, the flap 22 includes a fastener 50, such as a snap fastener that allows the flap 22 to be removably attached to the housing 21. In this way, the flap 22 encloses the housing 21. Other suitable fasteners are alternatively used in other embodiments, which include, but are not limited to: zip fasteners, button fasteners, and hook and loop fasteners.

The housing 21 further includes a handle 23 on the upper end 51 of the housing 21. The handle 23 is preferably U-shaped, wherein a first end 24 and a second end 25 of the handle 23 is affixed to the housing 21. The handle 23 is configured to be hand gripped by a user to allow for easy transport. It is anticipated that those of ordinary skill in the art will readily envision other suitable handles to the feminine hygiene system 20. These alterations and modifications are likewise contemplated.

The interior volume of the housing 21 is configured to support a reservoir 26 therein. The reservoir 26 is configured to support water or other suitable cleaning solutions therein. The reservoir 26 is preferably composed from a thermoset or thermoplastic material, however, other flexible and waterproof material are alternatively used in other embodiments. As illustrated, the reservoir 26 includes an inlet valve 27 that allows the reservoir 26 to be filled and refilled with water or other suitable cleansing fluids therein. The inlet valve 27 is configured to be enclosed by a cap 35 that prevents cleansing fluid supported within the reservoir 26 from flowing out of the water inlet valve 27. Preferably, the inlet valve 27 is removably secured via threaded elements on said cap 35.

The interior volume is also configured to support a hose 28. The hose 28, as illustrated, is placed in contact with the reservoir 26 to support the hose 28 within the interior volume of the housing 21. In some embodiments, the interior volume includes a separate compartment, wherein the hose 28 is configured to be received and supported therein. The hose 28 is preferably elongated in length and includes an attachment end 29 and a dispersing end 30. Preferably, the hose 28 is composed from rubber, however, other suitable material for transporting fluid therethrough are likewise contemplated.

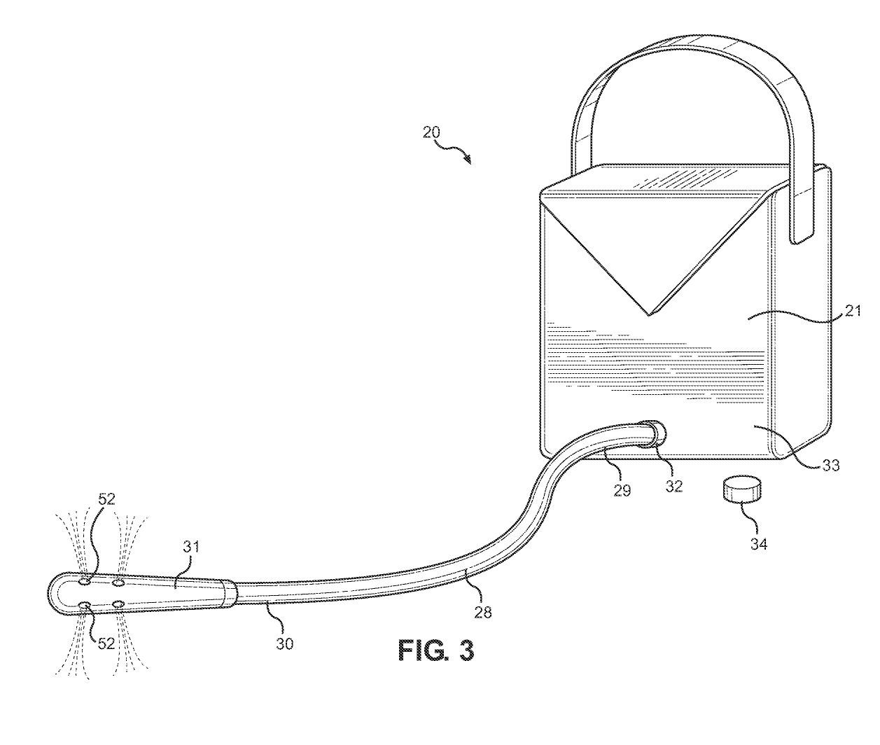

Referring now to FIG. 3, there is shown a front perspective of the feminine hygiene system 20. As depicted, the hose 28 is removably attached to the housing 21. The hose 28 includes an attachment end 29 and a dispersing end 30, wherein the attachment end 29 is configured to removably attach to an outlet valve 32 affixed to a lower end 33 of the housing 21. Preferably, the attachment end 29 is removably attached to the outlet valve 32 via threaded elements. The outlet valve 32 is configured to be in water communication with the reservoir to allow fluid from the reservoir to flow through the hose 28 and towards the dispersing end 30. The dispersing end 30 includes a nozzle 31 that allows for the fluid supported within the reservoir 26 to be dispersed. In this way, the user can use the fluid as needed. The nozzle is substantially tubular in shape and includes a plurality of apertures 52 disposed thereon configured to disperse cleansing fluid in different directions. The outlet valve 32 is also configured to be enclosed by a cap 34 when the attachment end 29 of the hose 28 is not removably attached.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1. A feminine hygiene system, comprising:
a housing having an upper end and a lower end forming an interior volume;
said interior volume configured to support a reservoir therein;
said reservoir configured to support a volume of cleansing fluid therein;
an inlet valve disposed on said reservoir, wherein said inlet valve is configured to allow said reservoir to be filled and refilled with said cleansing fluid;
said lower end of said housing comprising an outlet valve configured to be in fluid communication with said reservoir;
a hose configured to removably attach to said outlet valve to disperse said cleansing fluid.

2. The feminine hygiene system of claim 1, wherein:
said hose includes an attachment end and a dispersing end;
wherein said attachment end is configured to removably attach to said outlet valve;
said dispersing end includes a nozzle configured to disperse said cleansing fluid.

3. The feminine hygiene system of claim 1, wherein said housing is substantially rectangular in shape and resembles a purse.

4. The feminine hygiene system of claim 1, wherein said upper end includes a flap configured to enclose said interior volume via a fastener.

5. The feminine hygiene system of claim 4, wherein said fastener includes a snap fastener.

6. The feminine hygiene system of claim 1, wherein said flap is substantially V-shaped.

7. The feminine hygiene system of claim 1, wherein said upper end further includes a handle.

8. The feminine hygiene system of claim 1, wherein said handle is substantially U-shaped.

9. The feminine hygiene system of claim 1, further comprising a cap configured to removably enclose said inlet valve.

10. The feminine hygiene system of claim 1, further comprising a cap configured to removably enclose said outlet valve.

11. The feminine hygiene system of claim 1, wherein said housing is composed from waterproof material.

12. The feminine hygiene system of claim 1, wherein said reservoir is composed from a thermoset material.

13. The feminine hygiene system of claim 1, wherein said reservoir is composed from a thermoplastic material.

14. The feminine hygiene system of claim 2, wherein said nozzle is substantially tubular in shape.

15. The feminine hygiene system of claim 2, wherein said nozzle includes a plurality of apertures disposed thereon configured to disperse cleansing fluid in different directions.
